(19) United States
(12) Patent Application Publication
(10) Pub. No.: US 2011/0087037 A1
(43) Pub. Date: Apr. 14, 2011

(54) SYNTHESIS OF RING B ABEO-STEROLS AS NOVEL INHIBITORS OF MYCOBACTERIUM TUBERCULOSIS

(75) Inventors: Abimael D. Rodriguez-Pierluissi, San Juan, PR (US); Xiaomei Wei, Chicago, IL (US)

(73) Assignee: OFFICE OF INTELLECTUAL PROPERTY AND TECHNOLOGY VIC, San Juan, PR (US)

(21) Appl. No.: 12/740,901

(22) PCT Filed: Oct. 31, 2008
(86) PCT No.: PCT/US2008/081956

§ 371 Date: Dec. 9, 2010

Related U.S. Application Data
(60) Provisional application No. 60/983,990, filed on Oct. 31, 2007.

(51) Int. Cl. C07J 9/00

(52) U.S. Cl. 552/552

(57) ABSTRACT

Novel 3β-hydroxy-Δ⁵-steroid analogs possessing a contracted cyclopentane B-ring provide good inhibitory activity against Mycobacterium tuberculosis. The 5(6→7)abeo-sterol nucleus present in compounds of the invention represents a novel scaffold for the development of new antitubercular agents.

### GOVERNMENT INTEREST

The claimed invention was made with U.S. Government support under grant number S06GM08102 awarded by the National Institutes of Health (NIH). The government has certain rights in this invention.

### BACKGROUND ART

Tuberculosis is a common and deadly infectious disease caused by mycobacteria, mainly Mycobacterium tuberculosis. Tuberculosis most commonly attacks the lungs (as pulmonary TB) but can also affect the central nervous system, the lymphatic system, the circulatory system, the genitourinary system, bones, joints and even the skin. Over one-third of the world's population has been exposed to the TB bacterium, and new infections occur at a rate of one per second. In 2004, mortality and morbidity statistics included 14.6 million chronic active TB cases, 8.9 million new cases, and 1.6 million deaths, mostly in developing countries. In addition, a rising number of people in the developed world are contracting tuberculosis because their immune systems are compromised by immunosuppressive drugs, substance abuse, or HIV/AIDS. The rise in HIV infections and the neglect of TB control programs have enabled a resurgence of tuberculosis. The emergence of drug-resistant strains has also contributed to this new epidemic with, from 2000 to 2004, 20% of TB cases being resistant to standard treatments and 2% resistant to second-line drugs.

Multidrug-resistant TB (MDR TB) is TB that is resistant to at least two of the best anti-TB drugs, isoniazid and rifampicin. These drugs are considered first-line drugs and are used to treat all persons with TB disease. Extensively drug resistant TB (XDR TB) is a relatively rare type of MDR TB. XDR TB is defined as TB which is resistant to isoniazid and rifampin, plus resistant to any fluoroquinolone and at least one second-line drug.

There are several drugs approved by the United States Food and Drug Administration (FDA) for treating tuberculosis. In addition, the fluoroquinolones, although not approved by the FDA for tuberculosis, are used relatively commonly to treat tuberculosis caused by drug-resistant organisms or for patients who are intolerant of some of the first-line drugs. Rifabutin, approved for use in preventing Mycobacterium avium complex disease in patients with HIV infection but not approved for tuberculosis, is useful for treating tuberculosis in patients concurrently taking drugs that have unacceptable interactions with other rifamycins. Amikacin and kanamycin, nearly identical aminoglycoside drugs used in treating patients with tuberculosis caused by drug-resistant organisms, are not approved by the FDA for tuberculosis. Of the approved drugs, isoniazid (INH), rifampin (RIF), ethambutol (EMB), and pyrazinamide (PZA) are considered first-line anti-tuberculosis agents and form the core of traditional initial treatment regimens. Rifabutin and rifapentine may also be considered first-line agents under some specific situations. The remaining drugs are reserved for special situations such as drug intolerance or resistance.

| First line drugs | Second-line drugs |
|---|---|
| Isoniazid | Cycloserine |
| Rifampin | Ethionamide |
| Rifapentine | Levofloxacin* |
| Rifabutin* | Moxifloxacin* |
| Ethambutol | Gatifloxacin* |
| Pyrazinamide | p-Aminosalicylic acid |
| | Streptomycin |
| | Amikacin/kanamycin* |
| | Capreomycin |

*Not approved by the Food and Drug Administration for use in the treatment of tuberculosis.

### First-Line Drugs

Isoniazid (INH)—a first-line agent for treatment of all forms of tuberculosis caused by organisms known or presumed to be susceptible to the drug. It has profound early bactericidal activity against rapidly dividing cells.

Rifampin (RIF)—a first-line agent for treatment of all forms of tuberculosis caused by organisms with known or presumed sensitivity to the drug. It has activity against organisms that are dividing rapidly (early bactericidal activity) and against semidormant bacterial populations, thus accounting for its sterilizing activity. Rifampin is an essential component of all short-course regimens.

Rifabutin—is used as a substitute for RIF in the treatment of all forms of tuberculosis caused by organisms that are known or presumed to be susceptible to this agent. The drug is generally reserved for patients who are receiving any medication having unacceptable interactions with rifampin or have experienced intolerance to rifampin.

Rifapentine—may be used once weekly with INH in the continuation phase of treatment for HIV-seronegative patients with noncavitary, drug-susceptible pulmonary tuberculosis who have negative sputum smears at completion of the initial phase of treatment.

Pyrazinamide (PZA)—a first-line agent for the treatment of all forms of tuberculosis caused by organisms with known or presumed susceptibility to the drug. The drug is believed to exert greatest activity against the population of dormant or semidormant organisms contained within macrophages or the acidic environment of caseous foci.

Ethambutol (EMB)—a first-line drug for treating all forms of tuberculosis. It is included in initial treatment regimens primarily to prevent emergence of RIF resistance when primary resistance to INH may be present. Ethambutol is generally not recommended for routine use in children whose visual acuity cannot be monitored. However, if a child has adult-type tuberculosis or disease that is suspected or proven to be caused by organisms that are resistant to either INH or RIF, EMB should be used.

### Second-Line Drugs

Cycloserine—a second-line drug that is used for treating patients with drug-resistant tuberculosis caused by organisms with known or presumed susceptibility to the agent. It may also be used on a temporary basis for patients with acute hepatitis in combination with other nonhepatotoxic drugs.

Ethionamide—a second-line drug that is used for patients with drug-resistant tuberculosis disease caused by organisms that have demonstrated or presumed susceptibility to the drug.

Streptomycin (SM)—Its use and EMB have been shown to be approximately equivalent when used in the initial phase of treatment with 6-month regimens. However, among patients likely to have acquired M. tuberculosis in a high-incidence country, the relatively high rate of resistance to SM limits its usefulness.

Amikacin/Kanamycin—are two closely related injectable second-line drugs that are used for patients with drug-resistant tuberculosis whose isolate has demonstrated or presumed susceptibility to the agents. There is nearly always complete cross-resistance between the two drugs, but most SM-resistant strains are susceptible to both. Because it is used to treat a number of other types of infections, amikacin may be more easily obtained, and serum drug concentration measurements are readily available.

Capreomycin—a second-line injectable drug that is used for patients with drug-resistant tuberculosis caused by organisms that have known or presumed susceptibility to the drug.

p-Aminosalicylic acid (PAS)—an oral agent used in treatment of drug-resistant tuberculosis caused by organisms that are susceptible to the drug.

Fluoroquinolones—Of the fluoroquinolones, levofloxacin, moxifloxacin, and gatifloxacin have the most activity against M. tuberculosis. On the basis of cumulative experience suggesting a good safety profile with long-term use of levofloxacin, this drug is the preferred oral agent for treating drug-resistant tuberculosis caused by organisms known or presumed to be sensitive to this class of drugs, or when first-line agents cannot be used because of intolerance. Cross-resistance has been demonstrated among ciprofloxacin, ofloxacin, and levofloxacin and presumably is a class effect. Fluoroquinolones should not be considered first-line agents for the treatment of drug-susceptible tuberculosis except in patients who are intolerant of first-line drugs.

Antituberculous medications may have significant adverse effects. Thus, all patients taking TB therapy should be monitored periodically with a symptom review to assess possible toxicity. The most important adverse reactions reported for some commonly used anti-TB medications are summarized in the table below as reported in Interventions for Tuberculosis Control and Elimination, 2002. International Union Against Tuberculosis and Lung Disease.

| | Frequent (≧5 per 100 | Common (≧1-5 per 100 | Infrequent (≧1 per 1,000 |
|---|---|---|---|
| Drug | patients) | patients) | patients and <1 per 100 patients) |
| Isoniazid | Liver enzyme | | Hepatitis |
| | elevations | | Peripheral neuropathy |
| | | | Drug fever |
| Rifampin | Bilirubin elevations in | | Hepatitis |
| | the beginning of treatment | | Pruritus |
| | Orange discoloration | | Flu syndrome |
| | of urine and tears | | Drug fever |
| | Liver enzyme elevations | | |
| Pyrazinamide | Arthralgias | Nausea | Hepatitis |
| | | | Rash |
| | | | Nausea |
| Ethambutol | | | Retrobulbar neuritis |
| | | | Periaxial ocular toxicity |
| Streptomycin | Vestibular toxicity | Cochlear toxicity | Renal damage |
| | | Hypersensitivity | |
| | | reactions | |

### SUMMARY OF THE INVENTION

The present invention provides a novel method of synthesizing new abeo-sterol analogs showing inhibition against M. tuberculosis in an efficient, fast and inexpensive manner.

According to an aspect of the invention, the novel abeo-sterol analogs are synthesized from inexpensive known sterols.

In accordance with a further aspect of the invention, the novel abeo-sterol analogs show a high level of inhibition with low levels of toxicity in comparison to similar anti-tubercular agents.

Other features and advantages of the invention will be apparent from the following detailed description, and from the claims.

### BRIEF DESCRIPTION OF DRAWINGS

The foregoing and other objects, aspects and advantages of the invention will be better understood from the following detailed description of the preferred embodiments of the invention with reference to the accompanying drawings, in which:

FIG. 1 shows a naturally occurring steroid according to the invention.

FIG. 2 shows a naturally occurring abeo-sterol according to the invention.

FIG. 3 shows the structures of sterols and abeo-sterol analogs according to the invention.

### DISCLOSURE OF THE INVENTION

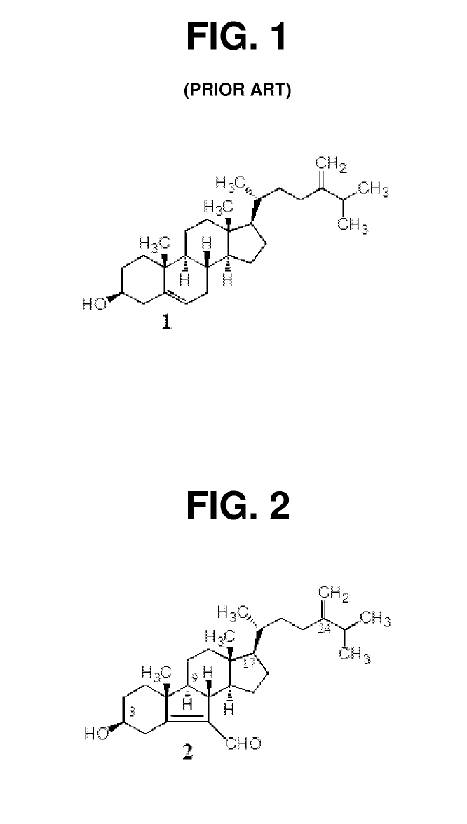

During a recent investigation, while searching for novel highly-active antitubercular natural products from the Caribbean Sea sponge Svenzea zeal, a complex mixture of sterols were isolated the structures of which, after careful purification, were confidently assigned by combined spectroscopic methods. Thus, the known sterol shown in FIG. 1 was obtained as the main isolate (2.2% yield) from the same hexane-soluble fractions as minor sterol shown in FIG. 2, which was isolated only in 0.007% yield. The MIC values for antitubercular activity of the compounds shown in FIG. 1 and FIG. 2 were determined as 120.1 and 7.8 μg/mL, respectively. Since the compound shown in FIG. 1, displaying only marginal activity against M. tuberculosis H37Rv, is a most plausible biosynthetic precursor to the sterol shown in FIG. 2, this finding suggested that in active steroids, maximum antimycobacterial activity could be attained upon contracting the cyclohexane B-ring, most likely as a result of increasing both the hydrophilic impact and rigidity of the steroidal backbone.

On the basis of reasonable activity shown by the sterol of FIG. 2, a small 5(6→7)abeo-sterol library was prepared for antimycobacterial screening by the Institute for Tuberculosis Research of the University of Illinois at Chicago. The significantly enhanced antitubercular activity suggested the importance of having a contracted cyclopentane B-ring in the steroid series described herein. Additionally, branching in the side chain at C₂₄ in combination with the 5(6→7)abeo-steroidal nucleus, appear to maximize the sterol's ability to be effective against M. tuberculosis.

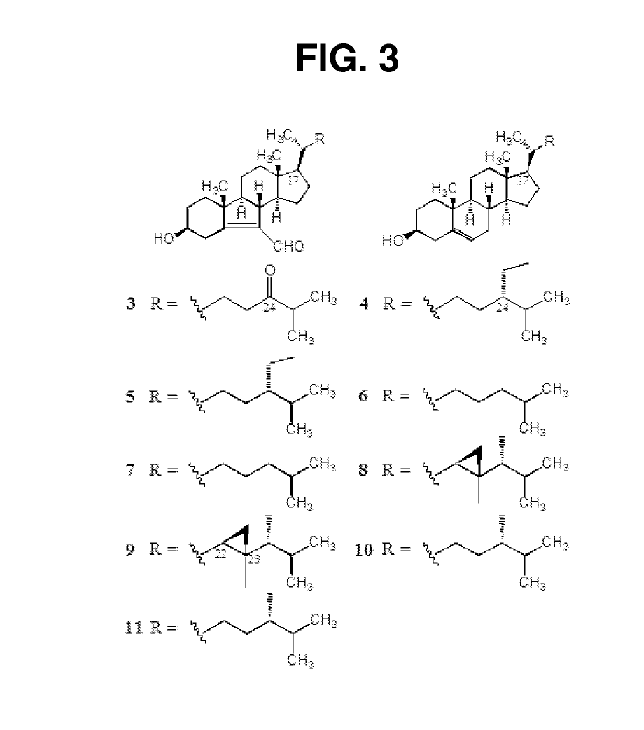

Five structurally diverse 5(6→7)abeo-sterols were synthesized for screening against M. tuberculosis H₃₇Rv (Mtb). While all of the sterol analogues synthesized 3, 5, 7, 9, and 11 shown in FIG. 3 possess the same tetracyclic 6-5-6-5 steroidal backbone, each analogue has a distinct alkyl side chain at C₁₇. For instance, the compound shown in FIG. 2 (the only naturally occurring steroid in this series) has a double bond in the side chain at C₂₄, whereas semi-synthetic sterols 3, 5, 9, and 11 possess either a carbonyl, a methyl or ethyl moiety at C₂₄. Analogue 9, on the other hand, displays further alkyl substitution at C₂₂ and C₂₃ of the side chain, whereas abeo-steroid 7 (prepared as a reference analogue from commercially available cholesterol) has no branching in the side chain at C₂₄. Interestingly, except for the latter analog, all of the abeo-sterols synthesized have shown ≧80% inhibitory activity against Mtb at a concentration of 8 μg/mL as shown on Table 1. On the other hand, none of the starting Δ⁵-unsaturated steroids 1, 4, 6, 8, and 10 display meaningful activity at 64 μg/mL.

The abeo-sterol analogs herein described were prepared in one-pot by the reactions of the corresponding 3β-hydroxy-Δ⁵-cholestanes with ozone at −78° C. in either Et₂O or CH₂Cl₂ solution after reductive work-up with dimethylsulfide under mild conditions. Intramolecular aldol condensation of the intermediate e-keto aldehydes (not isolated) under slightly basic conditions led to the desired abeo-sterols in a relatively low isolated yield, between 30 and 50%, depending on the purity of the starting sterols used. Final purification was subsequently achieved by silica gel flash column chromatography and the desired products thus obtained were used as such for rigorous structure characterization studies as well as antimycobacterial screening. The complete structural assignment of all of the synthetic abeo-steroid analogs described in the present invention was accomplished on the basis of comprehensive 1D and 2D NMR experiments involving ¹H-¹H COSY, DEPT, NOESY, ¹H-¹³C COSY (HMQC) and HMBC spectra, in addition to IR, UV, and HR-MS measurements. In most cases, the 2D NMR spectra provided both the structure and the complete and unambiguous proton and carbon atom assignments.

More specifically, the method can be described as follows. In a round bottom flask, a solution of chlolesterol in methylene chloride is ozonolyzed for 15 min at −78° C. After solvent removal, an ethereal solution of dimethyl sulfide is added and the resulting mixture is stirred at 25° C. overnight. Methanolic KOH is then added and the resulting solution is stirred for another 2 hours at 25° C. After removing the organic solvents in vacuo, the colorless oily residue left over is quickly purified by column chromatography using a short plug of silica gel yielding the corresponding 5(6→7)abeo-sterol in high yield.

TABLE 2
In vitro M. tuberculosis growth inhibition by sterols 1-11^{a}

| | % Inhibition | | | | | | | | Cyto-toxicity^{c} |
|---|---|---|---|---|---|---|---|---|---|
| | 128 | 64 | 32 | 16 | 8 | 4 | 2 | MIC^{b} | IC₅₀, μg/mL |
| Compds | μg/mL | | | | | | | μg/mL | (SI) |
| 1 | 97 | 40 | 43 | 33 | — | — | — | 120.1 | >128 (n.d.) |
| 2 | 99 | 99 | 99 | 105 | 91 | 69 | 52 | 7.8 | 51 (6.5) |
| 3 | 99 | 100 | 96 | 92 | 83 | 67 | 52 | 13.6 | 43.8 (3.2) |
| 4 | 45 | 48 | 31 | 32 | 7 | 8 | −1 | >128 | >128 (n.d.) |
| 5 | 99 | 99 | 99 | 98 | 99 | 97 | 29 | 3.8 | 26.6 (7) |
| 6 | 57 | 24 | 13 | 33 | — | — | — | >128 | >12.8 (n.d.) |
| 7 | 99 | 99 | 99 | 96 | 51 | 34 | 10 | 15 | >128 (>9) |
| 8 | 38 | 18 | 13 | 10 | — | — | — | >128 | >128 (n.d.) |
| 9 | 99 | 99 | 98 | 96 | 81 | 33 | 17 | 12.7 | 54.7 (4.3) |
| 10 | 58 | 41 | 31 | 21 | 23 | 14 | 15 | >128 | >128 (n.d.) |
| 11 | 99 | 100 | 99 | 99 | 97 | 94 | 39 | 3.9 | 423 (10.8) |
| RMP^{d} | 100 | 100 | 100 | 99 | 94 | 73 | 49 | 0.06 | 89.3 (1488) |

wherein, a indicates mean values of three experiments; b indicates the lowest drug concentration that effected an inhibition of ≧90% relative to untreated cultures; c indicates the Cytotoxicity against VERO cells (ATCC CCL-81), Selectivity index (SI)=IC₅₀/MIC, “n.d.” indicates not determined; and d indicates that Rifampin was used as a positive control.

Each of the starting 3β-hydroxy-Δ⁵-cholestanes 1, 4, 6, 8, and 10 and all of the synthesized 5(6→7)abeo-sterols 3, 5, 7, 9, and 11 were screened for their antimycobacterial activity. The activity results of steroids 1-11 are summarized in Table 1 above. The primary screen was conducted at 128, 64, 32, 16, 8, 4, and 2 μg/mL against Mtb H₃₇Rv (ATCC 27294) in BACTEC 12B medium using the Microplate Alamar Blue Assay (MABA). Because all of the synthesized abeo-sterols demonstrated 100% inhibition at ≧64 μg/mL, they were retested to determine the MIC, defined as the lowest concentration inhibiting growth by ≧90%. Rifampin (RMP) was used as a positive control during all the antituberculosis assays.

Concurrent with the determination of MICs, compounds 1-11 were tested for cytotoxicity (IC₅₀) in VERO cells in order to investigate the effect of contracting the cyclohexane B-ring on cytotoxic activity. The results described in Table 1 show that all of the starting steroids 1, 4, 6, 8, and 10 lacked significant cytotoxicity (IC₅₀'s>128 μg/mL). However, upon derivatization, analogues 3, 5, 9, and 11 showed some cytotoxicity (IC₅₀'s 26.6-54.7 μg/mL). Remarkably, the absence of appreciable toxicity in analogue 7 (IC₅₀>128 μg/mL) against mammalian cells in the VERO cell assay suggests that functionalization (branching or oxidation) at C₂₄ of the flexible alkyl side chain alone might explain the mild toxicity displayed by most abeo-sterols.

In conclusion, during a MABA bioassay against M. tuberculosis (H₃₇Rv), all of the abeo-sterol analogues synthesized showed minimum inhibitory concentrations of 3.8-15 μg/mL, while the starting 3β-hydroxy-Δ⁵ cholestanes had MIC values >128 μg/mL, respectively as shown in Table 1. The present invention suggests that the 5(6→7)abeo-steroidal nucleus inherently enhances antimycobacterial activity and thus represents a novel scaffold for the development of clinically useful agents for tuberculosis. Further correlations of structural features and the MICs of the six abeo-sterols scrutinized on this invention suggest that, in addition to the 5(6→7)abeo-steroidal moiety, the presence of a methyl or ethyl residue at C₂₄ is required for superior activity. The fact that modifications in the side chain can affect the antitubercular activity can be related to the permeability of the compounds through the Mtb membrane. Noteworthy is the activity of compound 3, which has two carbonyl groups and shows the worse selectivity index. Cytotoxicity results for a mammalian cell line indicate that in general strongly antitubercular abeo-sterols are also mildly cytotoxic. Although cytotoxicity of mammalian cells is low relative to toxicity to Mtb, it is not clear yet whether the observed activity of abeo-sterols is unique to mycobacterial targets. The present invention has demonstrated the future potential for development of B-ring abeo-sterol analogs as antimycobacterial agents and suggests new directions for the rational design of additional steroids that are active against the tubercle bacillus. In addition, the finding that abeo-sterol derivatives are inhibitors of Mtb is potentially very significant, as interest has been building recently in the metabolism of cholesterol by Mtb and the importance of such metabolism for intracellular survival in vivo.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of synthesizing abeo-sterol analogs comprising:
mixing a sterol compound with a first solvent;
reacting said mixture with ozone at a first predetermined temperature for a first predetermined amount of time;
removing said first solvent from said mixture;
stirring the resulting mixture with a mixture of an organosulfur compound and a second solvent at a second predetermined temperature for a second predetermined amount of time;
stirring the resulting mixture with a suitable base and a third solvent at a third predetermined temperature for a third predetermined amount of time;
removing the remaining solvents; and
purifying the final resulting mixture.

2. The method of claim 1, wherein said first predetermined temperature is about −78 Celsius degrees.

3. The method of claim 1, wherein said first predetermined amount of time is about 15 minutes.

4. The method of claim 1, wherein said first solvent comprises methylene chloride.

5. The method of claim 1, wherein said organosulfur compound comprises dimethyl sulfide.

6. The method of claim 1, wherein said second solvent comprises an ethereal solution.

7. The method of claim 1, wherein said second predetermined temperature is about 25 Celsius degrees.

8. The method of claim 1, wherein said second predetermined amount of time is not less than about 12 hours.

9. The method of claim 1, wherein said third predetermined temperature is about 25 Celsius degrees.

10. The method of claim 1, wherein said third predetermined amount of time is about 2 hours.

11. The method of claim 1, wherein said third solvent comprises a methanolic solution.

12. The method of claim 1, wherein said suitable base comprises KOH.

13. The method of claim 1, wherein removing the remaining solvents is done in vacuo.

14. The method of claim 1, wherein the final resulting mixture is purified by column chromatography.

15. The method of claim 1, wherein said abeo-sterols show inhibition against Mycobacterium tuberculosis.

16. An abeo-sterol analog especially suitable for inhibition against Mycobacterium tuberculosis having the formula: 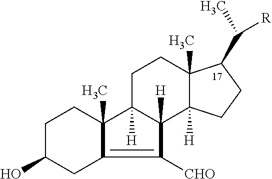
where R is an alkyl side chain.

17. The abeo-sterol of claim 16, wherein

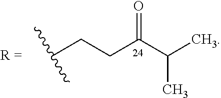

18. The abeo-sterol of claim 16, wherein

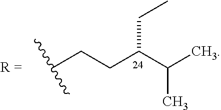

19. The abeo-sterol of claim 16, wherein

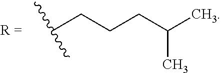

20. The abeo-sterol of claim 16, wherein

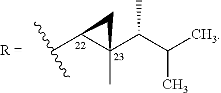

21. The abeo-sterol of claim 16, wherein

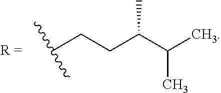

22. The abeo-sterol of claim 16, wherein said abeo-sterol is synthesized from a sterol having the formula: 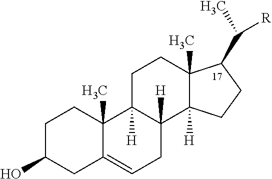
where R1 is an alkyl side chain.

23. The abeo-sterol of claim 22, wherein

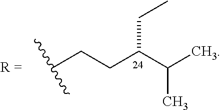

24. The abeo-sterol of claim 22, wherein

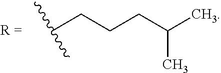

25. The abeo-sterol of claim 22, wherein

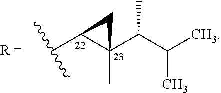

26. The abeo-sterol of claim 22, wherein [chemical structure]
